(19) United States
(12) Patent Application Publication
(10) Pub. No.: US 2025/0035613 A1
(43) Pub. Date: Jan. 30, 2025

(54) 3D BIOASSAYS TO MEASURE ANTIBODY-DEPENDENT CELL-MEDIATED CYTOTOXICITY

(71) Applicant: Wuxi Biologics Ireland Limited, Louth (IE)

(72) Inventors: Xiaoqing Jia, Shanghai (CN); Min Chen, Shanghai (CN)

(21) Appl. No.: 18/716,710

(22) PCT Filed: Nov. 24, 2022
(86) PCT No.: PCT/CN2022/133859

§ 371 Date: Jun. 5, 2024

(30) Foreign Application Priority Data
Dec. 6, 2021 (WO) PCT/CN2021/135837

(51) Int. Cl. G01N 33/50; G01N 33/68

(52) U.S. Cl. CPC G01N 33/5023; G01N 33/6857; G01N 2333/46; G01N 2333/70596; G01N 2333/7158; G01N 2333/775; G01N 2333/90241; G01N 2333/90287

(57) ABSTRACT

Provided herein are 3D bioassays to measure antibody-dependent cell-mediated cytotoxicity (ADCC). In particular, provided is a method for assessing ADCC comprising contacting an antibody or a functional antibody fragment thereof with a target cell and an effector cell expressing FcR, in a 3D cell culture under a condition allowing the interactions between those substances; and detecting the changes in the expression levels of biomarkers CXCL9, CXCL10 and CXCL11 and 0, 1, 2, 3, 4, 5, 6, 7, 8, 9 or 10 of the biomarkers selected from UBD, IDOL, STEAP4, JAG1, APOL4, GBP4, CD274, GBP5, CCL3 and CCL4; and assessing ADCC of the antibody based on the expression results. Also provided are the use and product relate to the method.

### TECHNICAL FIELD

The present disclosure concerns the field of bio-pharm analytics, clinical diagnosis and therapy. It inter alia pertains to 3D bioassays to measure antibody-dependent cell-mediated cytotoxicity (ADCC).

### BACKGROUND

Antibody-dependent cell-mediated cytotoxicity (ADCC), also called Antibody-dependent cellular cytotoxicity, is an immune mechanism through which Fc receptor-bearing effector cells can recognize and kill antibody-coated target cells expressing tumor- or pathogen-derived antigens on their surface. ADCC is one of major mode of action of many therapeutic antibodies (such as monoclonal and bispecific antibodies)^{[1]}. ADCC requires effector cells (e.g. NK cells, neutrophils, macrophages), target cells (e.g. solid tumor cells expressing the antigen of interest), and opsonizing antibodies^{[2]}. The engagement of FcγRIIlA on the effector cells with the opsonized target cells can triggers a cascade of signaling and biological events resulting in ADCC.

In brief, there are four main stages and mechanisms leading to the antibody-dependent effector-mediated killing of the target cell: (1) Recognition of the target cell and recognition of Fc receptor on the surface of the effector cell; (2) phosphorylation of immunoreceptor tyrosine-based activation motifs (ITAMs) by cellular src kinases within the effector cell; (3) triggering of three main downstream signaling pathways in the effector cell, resulting in cytotoxic granule polarization and release; and (4) killing of the target cell via the predominant perforin/granzyme cell death pathway.

Due to the importance of ADCC in therapeutic antibody clinical effects, many in vitro bioassays have been developed to determine the efficacy of antibodies in eliciting ADCC. Numerous associations between ADCC activity, Fc receptor polymorphisms, and clinical outcomes have been observed in both the settings of vaccination and monoclonal antibody therapy. Among those methods, the effector cells are usually peripheral blood mononuclear cells (PBMCs) or NK cells. And, the target cell lysis is usually determined by membrane integrity as a common endpoint, such as the assessment of enzymatic activity or the addition of a labeling step. Those in vitro methods are labor-intensive and are not necessarily correlated well to the in vivo or clinical effects, hence an unreliable predictor of in vivo drug efficacy^{[3]}.

The 3D cell culture is an artificial setting allowing the cells to grow and interact with each other and extracellular matrices in all three dimensions, like what they do in vivo^{[4]}. This is in contrast with traditional 2D cell cultures in which cells are grown in a flat monolayer on a plate. 3D cell cultures provide more accurate reflection of in vivo cell polarization, growth, differentiation, and survival^{[5,6]}.

Hence, there is a great need for novel, biomimic, reliable and sensitive assessments of antibody-dependent cell-mediated cytotoxicity effect and its control mechanism of therapeutic antibody.

### SUMMARY OF THE INVENTION

Here, we established a bioassay using 3D cell culture and carefully selected readout and excellent sensitivity to determination the dose dependent ADCC activity of therapeutic antibodies.

According to one aspect, disclosed herein is a method for assessing antibody-dependent cell-mediated cytotoxicity (ADCC), wherein the method comprises:
A) contacting (i) an antibody or a functional antibody fragment thereof; with (ii) a target cell; and (iii) an effector cell expressing FcR, in a 3D cell culture under a condition allowing the interactions between those substances;
B) detecting the changes in the expression levels of biomarkers CXCL9, CXCL10 and CXCL11 and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4; and
C) assessing ADCC of the antibody based on the result obtained in step B).

In some embodiments, the method can be used for but not limited to:
(a) screening a candidate antibody or antibody-binding fragment thereof or a conjugate comprising the antibody or the fragment based on the ability to induce ADCC against the target cell;
(b) controlling the quality of the antibody or the functional antibody fragment thereof, such as during the manufacture, storage, transportation and/or application of the antibody or the functional antibody fragment thereof;
(c) screening a candidate compound for the ability to modulate ADCC, wherein the method further comprises adding the candidate compound into the system of step A), and wherein the up-regulation in the expression level of the biomarkers, compared to the expression level determined without adding the candidate compound, is indicative of the ability of the candidate compound to up-modulate the ADCC function against the target cells;
(d) optimizing the types and/or concentration of the antibody or the functional antibody fragment thereof in inducing ADCC to the target cell;
(e) predicting the effect of the antibody or the function antibody fragment thereof in treatment of target cell associated diseases; and/or
(f) the interactions between two or more antibodies and/or functional antibody fragments thereof in ADCC, wherein the two or more antibodies and/or functional antibody fragments thereof are used in step A).

In one aspect, provided herein is a kit comprising agents for detecting the expression levels of CXCL9, CXCL10 and CXCL11, and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4.

In some embodiments, the kit further comprises one or more of 3D cell culture device and/or reagents, peripheral blood collection or effector cell isolation device, antibody or functional antibody fragments thereof.

In one aspect, provided herein is a use of substance(s) for determining the expression levels of CXCL9, CXCL10 and CXCL11, and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4, in the preparation of a product (such as a kit) for assessing ADCC.

In one aspect, provided herein is a use of substance(s) for use in the method of the present invention in the preparation of a kit for assessing ADCC.

In another aspect, provided herein is a system for assessing antibody-dependent cell-mediated cytotoxicity (ADCC), comprising:
a) means for contacting (i) an antibody or a functional antibody fragment thereof; with (ii) a target cell; and (iii) an effector cell expressing FcR, in a 3D cell culture under a condition allowing the interactions between those substances; and
b) means for determining the expression levels of biomarkers CXCL9, CXCL10 and CXCL11 and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: ADCC induced by anti-CD20 antibody in an assay system comprising PBMCs and Raji cells determined via conventional DELFIA labeling assay.

FIG. 2: gene expression during anti-CD20 antibody induced ADCC based on 3D ADCC assay, in which gene expression levels of particular biomarkers (i.e., CXCL11, CXCL9, CXCL10, UBD, IDO1, STEAP4, JAG1, APOL4, GBP4, CD274, GBP5, CCL3 and CCL4) are determined during an effective ADCC responses based on 3D ADCC assay.

FIG. 3: select chemokine expression level during anti-CD20 antibody induced ADCC based on 3D ADCC assay, in which expression levels of particular biomarkers CXCL11, CXCL9 and CXCL10 are determined during an effective ADCC responses based on 3D ADCC assay.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Current ADCC assays use 2D cell culture and they measure the target lyses as a common endpoint using different techniques (e.g. release of specific enzymes, or fluorescence or radioactive labeling). These assays are complicate in procedure with intrinsic variability and are not necessarily correlated to the in vivo or clinical responses of the same antibody product. Here we invented a bioassay that performed better in the 3D culture than the conventional 2D cell culture. We carefully selected the genotypical and protein readout and made the assay very sensitive and product specific. This 3D bioassay using PBMC and target cells can assess the ADCC activity of various antibodies (such as therapeutic recombinant monoclonal antibody). It is an in vitro bioassay, but it can offer results much better reflective of the in vivo clinical effect of antibody (such as a therapeutic antibody) than the conventional ADCC assays currently used in biopharma labs as well as clinical labs.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that other elements may be present.

The term “isolated” refers to a material that is substantially or essentially free from components that normally accompany it in its native state. The material can be a cell or a macromolecule such as a protein or nucleic acid. For example, an “isolated cell,” as used herein, refers to a cell, which has been purified from the cells in a naturally-occurring state.

### Antibody, Target Cell and Effector Cell

Provided herein is a novel in vitro system and method for assaying ADCC induced by an antibody or functional antibody fragment thereof to a target cell in the presence of an effector cell. As indicated above, the term “a” or “an” is intended to mean “one or more” (i.e., at least one) of the antibody or functional antibody fragment thereof, target cells and/or effector cells. The system and method can be used in a high throughput assay of ADCC.

As used herein, the term “antibody or functional antibody fragment thereof” refers to intact molecules as well as to fragments thereof, such as Fab, F(ab′)2 and Fv, which are capable of binding to the target cells. By “binding to target cells” is meant that an antibody is immuno-specific for the target cells, e.g., an antigen on the surface of the target cells. The term “immuno-specific” means that the antibody has substantially greater affinity for the antigen on the target cell than affinity for other proteins (e.g., other related proteins).

The antibody can be a known antibody that can induce ADCC or a new or candidate antibody whose effect in inducing ADCC is unknown and to be determined. The antibody or candidate antibody used in the methods provided herein can be, for example, a monoclonal antibody, a chimeric antibody, a humanized antibody, or a human antibody. Antibodies that can be used in the methods described herein also include antibodies that have been identified as having therapeutic potential (e.g., antibodies that have already undergone clinical trials).

The target cells useful herein can be Any suitable target cell can be selected and used according to the need in practice or to meet the assay of the antibody. Preferably, the target cell is capable of being specifically recognized and bound by the antibody or the functional antibody fragment thereof, for example the target cell expresses antibody specific antigenic epitope on its surface.

In some embodiments, the target cell is a diseased cell or cell line, such as a cancer cell or cell line, an infected cell or cell line (e.g., infected by a virus, bacterial, mycoplasma, chlamydia), a genetically defective cell or cell line. These target cells may be cell lines obtained from cell line banks. Alternatively, the cells can be obtained from an individual having a disease or a disorder. For example, target cells can be obtained from a tumor biopsy of a cancer patient.

Cancer cells that can be used as target cells include, but are not limited to, cells associated with Hodgkin's Disease, non-Hodgkin's B-cell lymphomas, T-cell lymphomas, malignant lymphoma, lymphosarcoma leukemia, chronic lymphocytic leukemia, multiple myeloma, chronic myeloid leukemia, chronic myelomonocytic leukemia, myelodysplastic syndromes, myeloproliferative disorders, hypereosinophilic syndrome, eosinophilic leukemia, multiple myeloma, X-linked lymphoproliferative disorders, esophageal cancer, stomach cancer, colon cancer, colorectal cancer, pancreatic cancer and gallbladder cancer, cancer of the adrenal cortex, ACTH-producing tumor, bladder cancer, brain cancer (e.g., neuroblastomas and gliomas), Ewing's sarcoma, head and neck cancer (e.g., mouth cancer and larynx cancer), kidney cancer (e.g., renal cell carcinoma), liver cancer, lung cancer (e.g., small and non-small cell lung cancers), malignant peritoneal effusion, malignant pleural effusion, skin cancers (e.g., malignant melanoma, tumor progression of human skin keratinocytes, epithelial cell carcinoma, squamous cell carcinoma, basal cell carcinoma), mesothelioma, Kaposi's sarcoma, bone cancer (e.g., osteomas and sarcomas such as fibrosarcoma and osteosarcoma), cancers of the female reproductive tract (e.g., uterine cancer, endometrial cancer, ovarian cancer, and cervical cancer), breast cancer, prostate cancer, retinoblastoma, testicular cancer, and thyroid cancer. Cancer cell lines that can be used as target cells can be any immortalized cell lines obtained, for example, from a cell bank.

Virally-infected cells that can be used as target cells include but not limited to cells infected with coronavirus (such as SARS-CoV-2), Epstein Barr Virus, HIV, influenza virus, polio virus, hepatitis A virus, Hepatitis B virus, Hepatitis C virus, Varicella zoster virus, Rubella virus, measles virus, Herpes Simplex Virus, Dengue virus, papilloma virus, respiratory syncytial virus, or rabies virus.

The target cells used in the methods provided herein also can be healthy cells. ADCC may be involved in the killing of healthy cells in patients with autoimmune diseases such as autoimmune thyroid disorders, myasthenia gravis, rheumatoid arthritis, systemic lupus erythematosus, immune haemolytic anaemia.

In some embodiments, a combination of more than one type of target cell can be used to, for example, more closely replicate the situation in vivo where the antibody may target organs and tissues comprising several cell types. The target cells thus can include more than one cell line or can include cells from more than one tissue. In some embodiments, the target cells include two or more or three or more cell types.

The effector cells used in the methods provided herein typically are cells that express one or more Fcγ receptors. In some embodiments, the FcγR is selected from FcγRI (e.g., FcγRIa, FcγRIb and FcγRIc), FcγRII (e.g., FcγRIIa, FcγRIIb and FcγRIIc) and FcγRIII (e.g., FcγRIIIa or FcγRIIIb), preferably FcγRII (such as FcγRIIa). Preferably, the FcγR is FcγRIIIa.

Suitable effector cells include, but are not limited to, peripheral blood mononuclear cells (PBMCs), natural killer (NK) cells, monocytes, cytotoxic T cells, and neutrophils. In some embodiments, the effector cells used in the methods described herein are PBMCs. PBMCs are a mixture of monocytes and lymphocytes that can be isolated from whole blood using, for example, standard experimental protocols described in the art.

In some embodiments, the effector cells may comprise an antibody or a functional antibody fragment of thereof attached or conjugated to the surface of the cells.

In some embodiments, the cells further comprise one or more report genes selected from the group consisting of fluorescin (such as luciferase, GFP), β-galactosidase, secreted alkaline phosphatase (SEAP).

In some embodiments, the antibody, the effector cell and/or target cell can be obtained or derived from patient or from a healthy donor.

In some embodiments, the ratio of effector cells to target cells (E:T) is ranged from 200:1 to 5:1. For example, the ratio of effector cells to target cells (E:T) can be 100:1, 50:1 and 25:1. The E:T ratio can be adjusted by a person skilled in the art according to need in practice.

In some embodiments, the antibody or functional antibody fragment thereof is added in a concentration ranged from 1 ng/mL-100 μg/mL. For example, the antibody or functional antibody fragment thereof can be added at a concentration of between about 0.01 and about 100 μg/mL, between about 0.1 and about 50 μg/mL, or between about 1 and about 10 μg/mL. The concentration of the antibody or functional antibody fragment thereof can be adjusted by a person skilled in the art according to need in practice.

### 3D Cell Culture

In the method and system, a 3D cell culture is used so as to provide more accurate reflection of in vivo cell polarization, growth, differentiation, and survival.

3D cell cultures used herein can be grown with or without a supporting scaffold, i.e., scaffold 3D cell culture and scaffold-fee 3D cell culture. Another way for 3D cell culture is microfluidic organ-on-a-chip models.

The Rotary Cell Culture System (RCCS) is a new technology for growing anchorage dependent or suspension cells in the laboratory. The RCCS is a horizontally rotated, bubble free disposable culture vessel with diffusion gas exchange. The system provides a reproducible, complex 3D in vitro culture system with large cell masses. During cell growing the rotation speed can be adjusted to compensate for increased sedimentation rates. The unique environment of low shear forces, high mass transfer, and microgravity, provides very good cultivating conditions for many cell types, cell aggregates or tissue particles in a standard tissue culture laboratory. RCCS is preferred, especially when the manufacture of antibody is also in a rotary suspension system. In some embodiments, the rotary cell culture system is set at a rotation rate of 60˜200 rpm, such as 100˜150 rpm.

In some embodiments, conventional culture medium suitable for the target cell can be used as the culture and assay medium, for example, RPMI 1640 with or without FBS or 10% HI-FBS. In some embodiments, the incubation is carried out at humidified 37° C. and 5% CO₂ in incubator orbital shaker.

For scaffold-free 3D cell culture, cells can be grown without the presence of a supporting scaffold. Scaffold-free methods rely on cells to self-assemble into clusters or spheroids. Popular scaffold-free methods include: low-adhesion plates, which are plates coated with hydrophilic polymer to prevent cells from sticking to surface, allowing cells to cluster together and form their own extracellular matrix (ECM); micropatterned surfaces, which are plastic surfaces modified to provide a micropattern or microwells which induce cells to grow as clusters; and hanging drop, in which cells are placed in a suspended drop of medium, allowing cells to aggregate and form spheroids at the bottom of the droplet.

For 3D scaffold cell culture, cells may be cultured within a supporting scaffold to allow growth in all directions. Popular types of scaffold include: hydrogels, which are polymeric materials containing a network of crosslinked polymer chains that can absorb and retain water derived from animals (such as Matrigel®, collagen) or plants (such as alginate/agarose), or synthesized from chemicals (such as QGel® Matrix); and inert matrices, such as sponge-like membranes made of polystyrene which contain pores for cells to proliferate and grow.

For microfluidic organ-on-a-chip models, an organ-on-a-chip is engineered to mimic the physiology of an organ. 3D cells are grown in scaffolds within the chambers of a microchip. Tiny channels allow the flow of liquid (microliter to picoliter volumes) to transport and distribute nutrients or other chemicals throughout the cells.

The 3D cell culture system disclosed herein provides more accurate reflection of in vivo cell polarization, growth, differentiation, and survival. For example, the 3D cell culture system disclosed herein can be particularly useful for Chemical Manufacturing and Control (CMC) for which bioassays are the only analytics to reflect the clinical efficacy of the drug and safety. Hence, the effective bioassay disclosed herein better reflective of the clinical efficacy of the drug is of great interest and beneficial.

### Biomarkers for ADCC and the Determination of the Expression Levels Thereof

In the present application, a group of biomarkers are used to determine the presence and/or extent of ADCC induced by antibody with effector cells to target cells.

The term “biomarker”, as used herein, may be interchangeably used with the term “endpoint”, which refers to a point marking the end of the assay with a definite effect is observed.

The expression levels and/or changes of the expression levels of biomarkers CXCL9, CXCL10 and CXCL11, as well as the expression levels and/or changes 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4 are determined to assess the presence and/or extent of ADCC. In some embodiments, the expression levels and/or changes thereof of 3, 4, 5, 6, 7, 8, 9, 10, 11, 12 or 13 of the above mentioned biomarkers are determined, wherein the biomarkers at least comprise CXCL9, CXCL10 and CXCL11.

In some embodiments, the biomarkers or the endpoints are selected by:
(1) isolating the whole mRNAs from the ADCC co-cultures;
(2) finding out the genes with most significant changes in the absence vs. presence of the antibody(ies) (such as via RNAseq technology); and
(3) selecting the genes whose expression levels change (up-regulated or down-regulated) most significantly as the biomarkers.

In some embodiments, the selection further comprises comparing the results obtained using a 2D cell culture with those of a 3D cell culture. For example, the selection may comprise:
(1′) isolating whole mRNAs from the ADCC co-cultures (2D vs. 3D);
(2′) finding out the genes with most significant changes in the absence vs. presence of the antibody(ies) (such as via RNAseq technology); and
(3′) selecting the genes upregulated most significantly in both 2D and 3D ADCC and in various parallel independent assays.

When comparing the results obtained from a 2D assay and a 3D assay, both assays show similar up-regulation or down-regulation trend for the same gene, however, the changes in the expression levels of the biomarkers observed in the 3D assays are more significant (for example, about 5-10 times higher than those observed in the 2D assay), which is indicative of a more sensitive and effective 3D assay for assessing ADCC in vivo.

The expression level can be either the mRNA expression level or the protein expression level. The expression level can be determined by but not limited to qRT-PCR, ELISA, Western Blot, HPLC, bioarray, and/or flow cytometry.

### Method for Determining ADCC and Use Thereof

Provided herein is an in vitro method using 3D cell culture and carefully selected readout and excellent sensitivity to determination the ADCC activity of the antibodies.

The ADCC is determined by a method comprising:
A) contacting (i) an antibody or a functional antibody fragment thereof; with (ii) a target cell; and (iii) an effector cell expressing FcR, in a 3D cell culture under a condition allowing the interactions between those substances;
B) detecting the changes in the expression levels of biomarkers CXCL9, CXCL10 and CXCL11 and 0, 1, 2, 3, 4, 5, 6, 7, 8, 9 or 10 of the biomarkers selected from UBD, TDO1, STEAP4, JAG1, APOL4, GBP4, CD274, GBP5, CCL3 and CCL4; and
C) assessing ADCC of the antibody based on the result obtained in step B).

Optionally, the method may further comprise determining whether the antibody or a functional antibody fragment thereof induces ADCC to the target cell, for example when it is not known whether the antibody can induce ADCC to the target cell. The determination can be carried out by but not limited to: fluorescein labeling assay (such as dissociation-enhanced lanthanide fluorescence immunoassay assay, DELFIA; Calcein AM labeling), enzyme labeling assay (such as lactose dehydrogenase assay), radioactive labeling assay (such as Cr⁵¹).

In step A), the target cell can be cultured or added into the 3D cell culture system before, simultaneously or after the addition of the effector cells and the antibody to the 3D cell culture system. Preferably, the target cell is cultured or added into the 3D cell culture system prior to the addition of the effector cells and the antibody.

Effector cells and antibody can be added to the target cell medium at the same time or separately. For example, effector cells can be added to the medium before antibody, or antibody can be added before effector cells. The methods described herein can employ any combination of target cells, effector cells, and antibodies, and the selection of target cells, effector cells, and antibodies used in the methods can depend on the purpose of the assay.

One or more types of antibodies or fragments thereof, one or more target cells and/or one or more effector cells can be added depending on the purpose of the assay.

In step B), the expression levels of the biomarkers and/or change of expression level before and after step A) or compared to a reference level can be determined. The determination can be carried out by but not limited to qRT-PCR, ELISA, Western Blot, HPLC, bioarray, and/or flow cytometry.

The reference level can be for example expression level of the biomarkers determined before step A) in the target cell culture; determined with an irrelevant antibody; or a standard level previous determined.

In step C), the ADCC effect of the antibody to the target cell can be assessed based on the expression level and/or the changes thereof determined in step B).

In some embodiments, the up-regulation in the expression level of the biomarkers, compared to a reference expression level, is indicative of the ability of the antibody or the functional antibody fragment to affect ADCC function against the target cells.

In some other embodiments, a down-regulation or no change in expression level of the biomarkers, compared to a reference expression level, is indicative of the inability of the antibody or the functional antibody fragment thereof to affect ADCC function against the target cells.

In some embodiments, the method can be carried out in a high-throughput way. For example, the method may be used in simultaneously assessing the ability of multiple combinations of antibodies, effector cells, and target cells to produce an ADCC response, optionally in the presence of compounds that may modulate the ADCC response.

### Use of the Method

The method of the present disclosure and the corresponding products (such as a kit for determining the synergistic ADE effect) are useful in laboratory, manufacture and clinic. For example, the bioassays invented can be carried out in the analytical labs in the biopharmaceutical company for product quality assessment during product development and manufacturing. They can also be carried out in the clinical labs to measure the clinical responses in a patient.

In some embodiments, the method of the present disclosure can be used in screening a candidate antibody or antibody-binding fragment thereof or a conjugate comprising the antibody or the fragment based on the ability to induce ADCC against the target cell. The candidate antibody or antibody-binding fragment thereof or a conjugate are added into the 3D cell culture system to contact with the target cell and the effector cells, and an up-regulation in the expression level of the aforementioned biomarkers, compared to a reference expression level, is indicative of the ability of the antibody or the functional antibody fragment or conjugate to affect ADCC function against the target cells, and vise-versa.

In some embodiments, the method of the present disclosure can be used in controlling the quality of the antibody or the functional antibody fragment thereof, such as during the manufacture, storage, transportation and/or application of the antibody or the functional antibody fragment thereof. For example, an abnormal expression level of the aforementioned biomarkers, compared to a reference expression level (such as level in a quality guarantee range), led by the antibody or antibody-binding fragment thereof is indicative of an unqualified antibody or the functional antibody fragment, and vise-versa.

In some embodiments, the method of the present disclosure can be used in screening a candidate compound for the ability to modulate ADCC, wherein the method further comprises adding the candidate compound into the system of step A). For example, the up-regulation in the expression level of the biomarkers, compared to the expression level determined without adding the candidate compound, is indicative of the ability of the candidate compound to up-modulate the ADCC function against the target cells, and vise-versa.

In some embodiments, the method of the present disclosure can be used in optimizing the types (including combinations) and/or concentration of the antibody or the functional antibody fragment thereof in inducing ADCC to the target cell. For example, in step A), various types (including combinations) and/or concentration of antibody or the functional antibody fragment thereof can be added, and the optimization can be made based on the expression levels of the biomarkers.

In some embodiments, the method of the present disclosure can be used in predicting the effect of the antibody or the function antibody fragment thereof in treatment of target cell associated diseases. For example, the up-regulation in the expression level of the biomarkers, compared to the expression level, is indicative of the ability of the antibody in inducing ADCC function against the target cells, and vise-versa. If ADCC is useful in treatment of the disease (such as cancer), the antibody has treatment effect to the disease. If ADCC is not benefit for the treatment of a disease (such as an autoimmune disease), the antibody is not suggested to be used.

In some embodiments, the method of the present disclosure can be used in assessing the interactions between two or more antibodies and/or functional antibody fragments thereof in ADCC, wherein the two or more antibodies and/or functional antibody fragments thereof are used in step A).

It is to be understood that this invention is not limited to the particular embodiments described herein and as such can vary. Those of skill in the art will recognize that there are numerous variations and modifications of this invention, which are encompassed within its scope.

### Corresponding Products and Systems

Also provided herein are products relating to the present methods and uses.

Provided herein is a kit comprising agents for detecting the expression levels of CXCL9, CXCL10 and CXCL11, and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4. The kit can be used in the method of the present disclosure in for example step B). The kit can also be used in other methods for ADCC assessment or as a supplementary means. The kit may further comprise one or more of 3D cell culture device and/or reagents, peripheral blood collection or effector cell isolation device, antibody or functional antibody fragments thereof.

Provided herein is a system for assessing antibody-dependent cell-mediated cytotoxicity (ADCC). The system may comprise:
a) means for contacting (i) an antibody or a functional antibody fragment thereof; with (ii) a target cell; and (iii) an effector cell expressing FcR, in a 3D cell culture under a condition allowing the interactions between those substances; and
b) means for determining the expression levels of biomarkers CXCL9, CXCL10 and CXCL11 and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4.

Optionally, the system may further comprising c) means for assessing ADCC of the antibody based on the change of the expression levels.

In some embodiments, the system may be an automated operating system.

Various device components can be components as generally known in the art. For example, control electronics may be provided in any suitable form and may, for example, include memory and a processor. Processor may one or more components that can include any one or more of a microprocessor, a controller, a digital signal processor (DSP), an application specific integrated circuit (ASIC), a field-programmable gate array (FPGA), equivalent discrete or integrated logic circuitry, programmable logic circuitry, or the like, and the functions attributed to processor herein may be embodied as hardware, firmware, software or any combination thereof. Memory may store instructions that cause processor to provide the functionality ascribed to programmer herein, and information used by processor to provide the functionality ascribed to programmer herein. Memory may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory may also store information that controls the parameters in the method (such as the rotary rate of the 3D cell culture system).

Such hardware, software, firmware may be implemented within the same device or within separate devices to support the various operations and functions described in this disclosure. In addition, any of the described units, modules or components may be implemented together or separately as discrete but interoperable logic devices. Functionality associated with one or more modules or units may be performed by separate hardware or software components, or integrated within common or separate hardware or software components.

The invention will be further described in the following example, which does not limit the scope of the invention described in the claims.

### EXAMPLES

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### Example 1. Conventional DELFIA Labeling Based ADCC Assay

Antibody: anti-CD20 therapeutic antibody (INV-0061401, Roche, China) were serially diluted in assay medium (RPMI 1640+10% HI-FBS) to obtain antibody solutions at different concentrations (6000, 2000, 666.67, 222.22, 74.07, 24.69 and 8.23 ng/mL, respectively).

Target cells: CD20-expressing Raji cells (CCL-86, ATCC, USA) were labeled with DELFIA BATDA (Cat #C136-100, Perkin Elmer, China) for 30-40 minutes at 37° C. in a humidified incubator set at 37° C. and 5% CO₂. After incubation, the Raji cells were centrifuged and washed with 100 mM sulfinpyrazone before resuspended in the assay medium (RPMI 1640+10% HI-FBS) to a density of 2×10\hat{ }5 cells/mL.

Effector cells: peripheral blood mononuclear cells (PBMCs) were isolated from whole blood of healthy donor by density gradient centrifugation and resuspended in the assay medium to a density of 8.5˜10×10\hat{ }6 cells/mL.

Co-culturing: 8.5˜10×10\hat{ }6 cells/mL PBMCs were mixed well with 2×10\hat{ }5 cells/mL DLFIA BATDA labeled CD20-expressing Raji cells in a volume ratio of 1:1. To a 96-well plate, the cell mixture was added (100 μL/well) and then anti-CD20 therapeutic antibody diluents (0˜3000 ng/mL, as prepared above; 100 μL/well). The 96-well plate was incubated for 2-4 hours in a humidified 37° C. and 5% CO₂ incubator.

Negative control (NC) well: a mixture of Raji cells and PBMC cells was added (baseline); Positive control well: a mixture of Raji cells, PBMC cells and cell lysis buffer was added in a relevant concentration of 10% (20 μL lysis mixture into 180 μL cell culture); Tₘₐₓ well: a mixture of Raji cells and cell lysis buffer was added; Tₘᵢₙ well: only Raji cells were added; Irrelevant control well: a mixture of Raji cells and PBMC cells with irrelevant anti-HER2 antibody was added (which shows a similar result to the NC well).

After plate centrifugation at 500×g for 3˜5 minutes, 20 μL of supernatant was taken from each well and transferred to corresponding wells in a 96-well white microwell plate (3912, Corning, USA). Subsequently, 200 μL of DELFIA Europium Solution (Cat #C135-100, Perkin Elmer, China) were added to each well and the plates were incubated for 15-20 minutes before acquired on a plate reader M5e (Molecular Devices, California, USA). The parameters for readout were set as follows:
Read Mode: Endpoint, top read
Ready Type: TRF
Wave length: Ex 345 nm, Em: 615 nm, Cutoff: 590 nm
Sensitivity: Readings: 100; PMT: Auto

Relative fluorescence units (RFU) were plotted against the antibody concentrations to generate a 4-parameter logistic response curve. Relative potency of the sample was calculated by EC₅₀ ratio when all system and sample suitability was met.

### Results

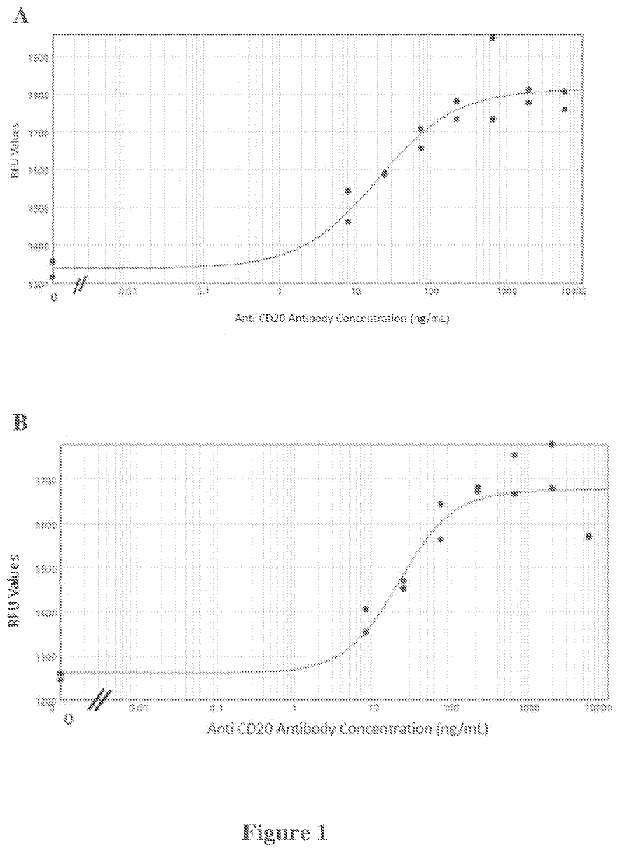

In this experiment, we used conventional target cell labeling method to evaluate if the anti-CD20 antibodies can induce an effective ADCC response with the PBMCs and Raji cells. FIGS. 1A and 1B are two independent assays using PBMCs from 2 healthy donors respectively (as effector cells), independently prepared Raji cells (as target cells) and anti-CD20 antibodies.

As shown in FIGS. 1A and 1B, in the absence of anti-CD20 antibody (and presence of irrelevant anti-HER2 antibody), there was no Raji cell lysis detected. In the presence of anti-CD20 antibody, there was a dose-dependent increase of Raji cell lysis, represented by RFU signals.

Our results also showed that the Raji cell lysis requires the presence of all 3 components: PBMC, Raji, and anti-CD20 antibodies. In the absence of any one of these components, there was no response in the assay. PBMCs from 2 healthy donors were tested in the assay and both were effective to induce an anti-CD20 Ab-dose dependent Raji cell lysis.

The results suggest that for the anti-CD20 antibody, ADCC is a major mode of action for the therapeutic antibody via the interactions between the antibody, the effector cells and the target cells.

### Example 2. ADCC-Indicating Endpoint Gene Selection

### RNA Extraction

Total RNA was extracted from the 10⁶ cells from the cell mixture of PBMC and Raji cells using TRIzol@ Reagent (Invitrogen, 15596026) and RNeasy minElute spin column (Qiagen, 74204) according to the manufacturer's instructions in Mingma Technologies Co., Ltd at Shanghai. Then the integrity of the total RNA was determined by 2100 Bioanalyser (Agilent) and quantified using the NanoDrop (Thermo Scientific). About 500-1000 ng high-quality RNA sample (OD_{260/280}=1.9˜2.0, RIN≥8) was used to construct sequencing library.

### Library Preparation and Illumina Sequencing

RNA purification, reverse transcription, library construction and sequencing were performed in Mingma Technologies Co., Ltd at Shanghai according to the manufacturer's instructions (Vazyme & Illumina). The mRNA-focused sequencing libraries from total RNA were prepared using VAHTS mRNA-seq v3 Library Prep Kit (Vazyme, NR611-01). PolyA mRNA was purified from total RNA using oligo-dT-attached magnetic beads and then fragmented by fragmentation buffer. Taking these short fragments as templates, first strand cDNA was synthesized using reverse transcriptase and random primers, followed by second strand cDNA synthesis. Then the synthesized cDNA was subjected to end-repair, phosphorylation and ‘A’ base addition according to Illumina's library construction protocol. Then Illumina sequencing adapters were added to the dscDNA fragments. After PCR amplification for DNA enrichment, the AMPure XP Beads (Beckmen, A63881) were used to clean up the target fragments of 200˜300 bp.

After library construction, Qubit 4.0 fluorometer dsDNA HS Assay (Thermo Fisher Scientific, Q32854) was used to quantify concentration of the resulting sequencing libraries, while the size distribution was analyzed using Agilent BioAnalyzer 2100 (Agilent).

Sequencing was performed using an Illumina system following Illumina-provided protocols for 2×150 paired-end sequencing in Mingma Technologies Co., Ltd at Shanghai.

Qualified data were used to study the gene expression relative to a house keeping gene (GAPDH, β2m, or 18S). The relative gene expression was measured by the expression of the target gene divided by that of the house keeping gene (i.e. Ct of the target gene subtracted from the Ct of the house keeping gene). The relative amount of antibody-stimulated group gene expression was calculated by dividing the same gene expressed by the NC group on the same plate.

Among 57730 genes, thirteen particular genes (i.e., CXCL1152 CXCL96 CXCL10, UBD, IDO1, STEAP4, JAG1, APOL4 GBP4, CD274, GBP5, CCL3 and CCL4) were found significantly upregulated in the ADCC group compared to the isotype control group. They were expressed in both 2D ADCC and 3D ADCC with the about 5 to about 10 times (or even higher) more expression in the latter. Therefore, those genes were selected as the biomarkers for ADCC assessment.

### Gene Expression Level of Exeriment 1 (Donor 1)

| [Anti-CD20 | | | | | | |
|---|---|---|---|---|---|---|
| Ab] | ADCC 2D | | | ADCC 3D | | |
| (ng/mL) | 0 | 6000 | 300 | 0 | 6000 | 300 |
| CXCL11 | 1 | 0 | 0 | 1 | 366.1048 | 302.1518 |
| CXCL9 | 1 | 14.77708 | 16.87728 | 1 | 187.2544 | 152.4969 |
| CXCL10 | 1 | 4.909889 | 5.464928 | 1 | 85.1199 | 69.99793 |
| GBP5 | 1 | 2.968041 | 3.521379 | 1 | 13.08827 | 11.66677 |
| GBP4 | 1 | 3.935401 | 4.724835 | 1 | 14.29991 | 12.97079 |
| IDO1 | 1 | 5.04905 | 5.72894 | 1 | 76.521 | 60.3303 |
| UBD | 1 | 0 | 0 | 1 | 78.3406 | 73.5616 |
| STEAP4 | 1 | 0 | 0 | 1 | 51.9283 | 41.7432 |
| APOL4 | 1 | 0 | 0 | 1 | 22.132 | 19.6956 |
| JAG1 | 1 | 0 | 0 | 1 | 22.4659 | 21.7006 |
| CCL4 | 1 | 3.21197 | 3.32734 | 1 | 10.3136 | 8.89229 |
| CD274 | 1 | 2.41335 | 2.97897 | 1 | 14.2133 | 12.2022 |
| CCL3 | 1 | 2.98436 | 2.91254 | 1 | 12.1276 | 10.7357 |

### Gene Expression Level of Experiment 2 (Donor 2)

| [Anti-CD20 | | | | | | |
|---|---|---|---|---|---|---|
| Ab] | ADCC 2D | | | ADCC 3D | | |
| (ng/mL) | 0 | 6000 | 300 | 0 | 6000 | 300 |
| CXCL11 | 1 | 21.98591 | 43.61159 | 1 | 142.4712 | 119.3137 |
| CXCL9 | 1 | 24.46462 | 42.04547 | 1 | 110.2654 | 93.46266 |
| CXCL10 | 1 | 8.29225 | 15.81956 | 1 | 23.39777 | 21.15824 |
| GBP5 | 1 | 6.339856 | 6.813381 | 1 | 13.00932 | 13.3797 |
| GBP4 | 1 | 6.043697 | 5.85465 | 1 | 11.35546 | 12.83608 |
| IDO1 | 1 | 3.865139 | 5.337133 | 1 | 33.96928 | 30.29844 |
| UBD | 1 | 0 | 0 | 1 | 31.10821 | 31.76052 |
| STEAP4 | 1 | 0 | 0 | 1 | 30.63752 | 26.22596 |
| APOL4 | 1 | 0 | 0 | 1 | 16.32322 | 14.86582 |
| JAG1 | 1 | 0 | 0 | 1 | 16.1264 | 13.56744 |
| CCL4 | 1 | 3.881519 | 4.115395 | 1 | 15.08093 | 12.81615 |
| CD274 | 1 | 5.932365 | 8.412392 | 1 | 12.75545 | 11.43196 |
| CCL3 | 1 | 3.577445 | 4.496143 | 1 | 12.74663 | 10.59718 |

### Example 3. Gene Expression Based 3D ADCC Assay

4.25˜5×10\hat{ }6 PBMC cells were incubated with 1×10\hat{ }5 Raji cells (in a volume ratio of 1:1) in the absence or presence of anti-CD20 antibody (final concentration: 6000, 300, 0 ng/mL) in the same assay medium as described above. Negative Control (NC) group has no anti-CD20 antibody. Instead, an isotype-matched irrelevant comparison anti-HER2 antibody was used in the NC group.

PBMCs, Raji cells, and antibodies were co-cultured in a 24 well deep well plate at 2000 μL per well in a humidified 37° C. and 5% CO₂ incubator orbital shaker (ISFT-XC, Kunner, Germany) for approx. 4 hours. The shaking speed was set at 120 rpm.

By comparison, 2D assays were carried out with equivalent amount of PBMCs, Raji cells, and anti-CD20 or anti-HER2 antibodies in a 24 well deep well plate in a humidified 37° C. and 5% CO₂ incubator shaker for ˜4 hours set at 0 rpm.

Following incubation, total RNA from each well was extracted using Trizol Reagent (15596026, Invitrogen, China) and RNeasy minElute spin column (74204, Qiagen, China). RNA purification, reverse transcription, library construction and sequencing were performed according to the manufacturer's instructions (Vazyme & Illumina). High-throughput sequencing was performed using Illumina system following standard protocol by Illumina with 2×150 paired-end sequencing.

Qualified data were used to study the gene expression of the 13 biomarkers selected according to Example 2 relative to a house keeping gene (GAPDH, β2m, or 18S). The relative gene expression was measured by the expression of the target gene divided by that of the house keeping gene (i.e. Ct of the target gene subtracted from the Ct of the house keeping gene). The relative amount of antibody-stimulated group gene expression was calculated by dividing the same gene expressed by the NC group on the same plate.

### Results

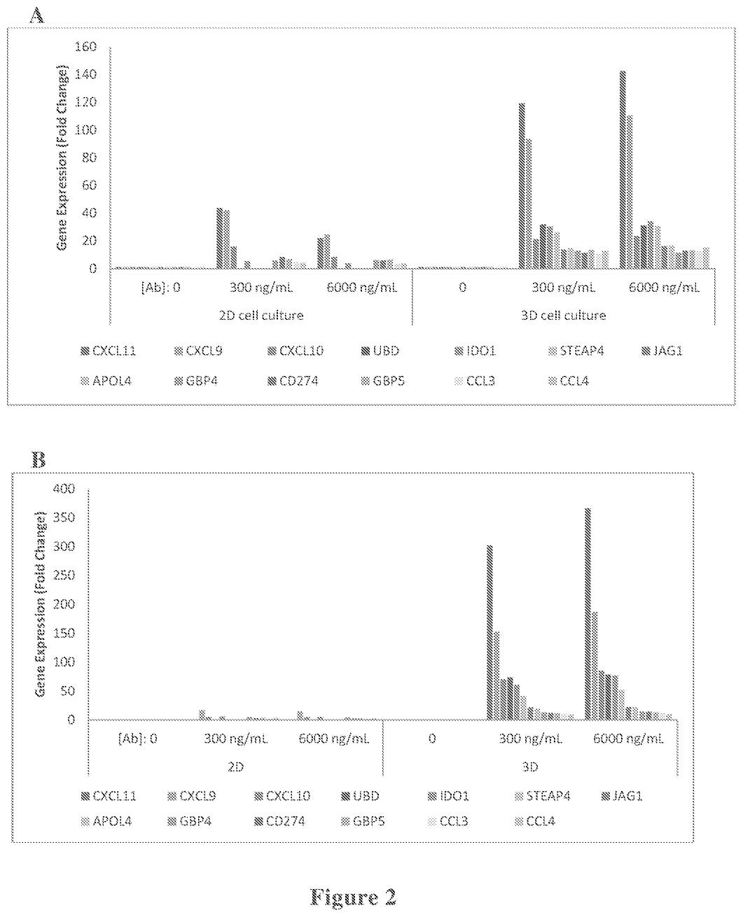

In this experiment, we used 24-well deep well plates to investigate the gene expression levels during an effective ADCC response. Two independent experiments were carried out using PMBCs from 2 healthy donors respectively and the results were shown in FIGS. 2A and 2B, respective.

Both conventional 2D cell culture and innovative 3D cell culture were used and compared. As shown in FIGS. 2A and 2B, 13 biomarker genes (i.e., CXCL11, CXCL9, CXCL10, UBD, IDO1, STEAP4, JAG1, APOL4, GBP4, CD274, GBP5, CCL3 and CCL4) significantly up-regulated in the presence of PBMC, Raji cells, and anti-CD20 antibody compared to the wells absent of anti-CD20 antibody. Most of the up-regulated genes are immune-regulatory and inflammatory-related genes. Gene up-regulations happened in both 2D and 3D cell cultures. However, 3D cell culture generated 5-10 times more significant up-regulation of the same genes compared to those in the 2D cell culture.

The results suggest that: from one aspect, 3D cell culture is a more suitable assay format to measure gene expression regulations (i.e., significantly up-regulated gene expression of one or more of the above particular biomarkers) during antibody specific ADCC. From the other aspect, the results also suggest that combining 3D cell culture with particular endpoints (i.e., significantly up-regulated gene expression of one or more of the particular biomarkers) whether an antibody can produce any ADCC effect and the strength of the ADCC can be determined.

### Example 4. Chemokine Expression Based 3D ADCC Assay

4.25˜5×10\hat{ }6 PBMC cells were incubated with 1×10\hat{ }5 Raji cells (in a volume ratio of 1:1) in the absence or presence of anti-CD20 antibody (6000, 300, 0 ng/mL) in the same assay medium as described above. The cells and antibodies were mixed in 24 well deep well plate at 2000 μL per well and co-cultured in a humidified 37° C. and 5% CO₂ incubator shaker (ISFI-XC, Kunner, Germany) for ˜4 hours set at 120 rpm.

By comparison, 2D assays were carried out with equivalent amount of PBMCs, Raji cells, and anti-CD20 or anti-HER2 antibodies in a 24 well deep well plate in a humidified 37° C. and 5% CO₂ incubator shaker for ˜4 hours set at 0 rpm.

Supernatant was collected and CXCL9, 10, and 1 levels were measured by ELISA kits (SEK10888, KIT10768, SEK10876, Sino Biological, China). Briefly, anti-CXCL9, anti-CXCL10, or anti-CXCL11 antibodies were coated onto the 96-well plate at 0.5-2 pg/mL respectively and 100 μL per well. After incubation for ˜15 hours at 2-8° C., the plate was blocked with blocking buffer for 1˜2 hours on an orbital shaker (New Brunswick Scientific/Innova 40R, US) set at 25° C. and 220 RPM.

Following incubation, the plate was washed 3 times with PBS/TBS containing 0.05% Tween 20. Recombinant chemokine standards were diluted in a dilution plate to 0-600 pg/mL (CXCL9), 0-250 pg/mL (CXCL10), or 0-800 pg/mL (CXCL11) and loaded to corresponding wells in the ELISA plate. 300 μL of supernatant was transferred to the 1^{st} row of a dilution plate, then 150 μL was transferred to the 2^{nd} row preloaded with 150 μL of Assay Buffer (1/2 dilution) and so on to prepare the neat, 1/2 dilution, 1/4 dilution, 1/8 dilution and 1/16 dilution samples. Then 100 μL of the supernatant samples were transferred to corresponding wells in the ELISA plate, incubated for 1˜2 hours at 25° C. and 220 RPM. After plate wash, 100 μL per well of HRP-conjugated anti-chemokine detection antibodies were added and incubated for ˜1 hours at 25° C. and 220 RPM.

After plate wash, 100 μL of TMB (5120047, KPL, USA) were added and incubated for ˜30 minutes at RT. The reaction was stopped by add 100 μL of 1N H₂SO₄. The OD signals for CXCL9, 10 and 11 were acquired from each well and analyzed by a SoftMax Pro template (Molecular Devices, California, USA) plotted against the anti-CD20 antibody concentrations. A 4-parameter logistic regression analysis was used to calculate the chemokine levels in the supernatant referred to the Standards.

The data was subsequently exported to SoftMax Pro software and plotted using 4-parameter fit analysis.

### Results

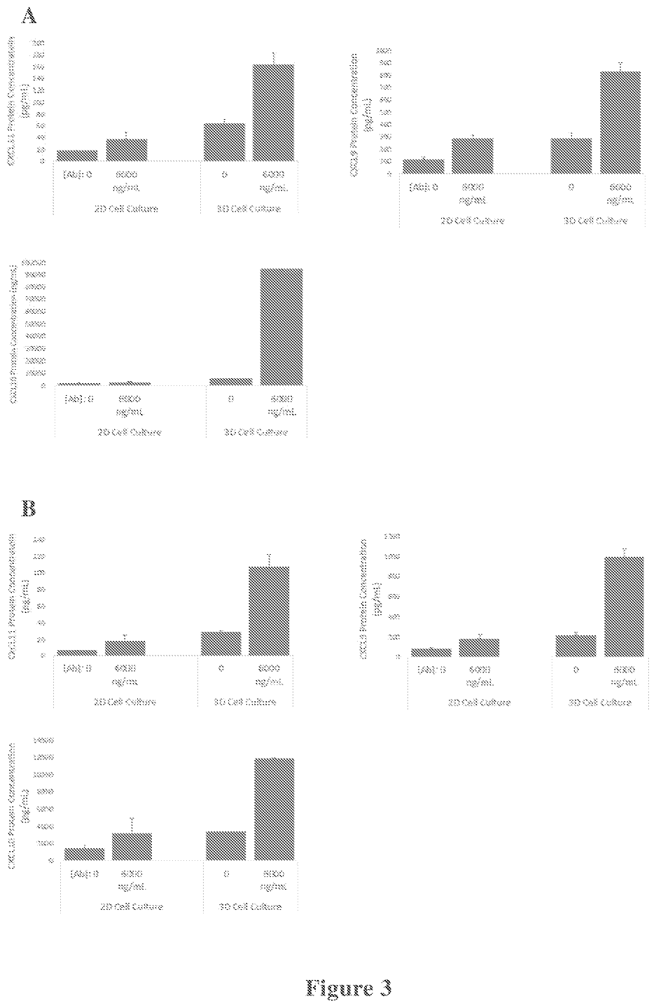

To further investigate if there are measurable and quantifiable changes in the protein expression during antibody specific ADCC, the select chemokine protein levels were measured by ELISA from the ADCC co-culture supernatant. Two independent experiments were carried out using PMBCs from 2 healthy donors respectively and the results were shown in FIGS. 3A and 3B, respectively.

Both conventional 2D cell culture and innovative 3D cell culture were used and compared. As shown in FIGS. 3A and 3B, consistent to the gene expression levels, 3D cell culture generated more significantly CXCL11, 9, and 10 protein expressions in the presence of PBMC, Raji cells, and anti-CD20 antibodies.

The results suggest that: from one aspect, 3D cell culture is a more suitable assay format to measure chemokine expression (i.e., significantly up-regulated chemokine expression of one or more of the above particular protein biomarkers) during antibody specific ADCC. From the other aspect, the results also suggest that combining 3D cell culture with particular endpoints (i.e., significantly up-regulated protein level of one or more of the particular biomarkers) can be used to effectively predict and/or determine whether an antibody can produce any ADCC effect and the strength of the ADCC.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### REFERENCES

1. Zahavi D, AlDeghaither D, O'Connell A, Weiner LM (2018). Enhancing antibody-dependent cell-mediated cytotoxicity: a strategy for improving antibody-based immunotherapy. Antib Ther. 1:7.
2. Wang W, Erbe A K, Hank J A, Morris Z S, Sondel P M (2015). NK Cell-Mediated Antibody-Dependent Cellular Cytotoxicity in Cancer Immunotherapy. Front Immunol. 6: 368.
3. Boero S, Morabito A, Banelli B (2015). Analysis of in vitro ADCC and clinical response to trastuzumab: possible relevance of FcγRIIIA/FcγRIIA gene polymorphisms and HER-2 expression levels on breast cancer cell lines J Transl Med. 13: 324.
4. Fey S, Wrzesinski K (2013). Determination of Acute Lethal and Chronic Lethal Thresholds of Valproic Acid Using 3D Spheroids Constructed From the Immortal Human Hepatocyte Cell Line HEPG2/C3A. In Boucher A (ed.). Valproic Acid. Nova Science Publishers, Inc. pp. 141-165.
5. Griffith L G, Swartz M A (2006). Capturing complex 3D tissue physiology in vitro. Nature Reviews Molecular Cell Biology. 7 (3): 211.
6. Pampaloni F, Reynaud E G, Stelzer E H (2007). The third dimension bridges the gap between cell culture and live tissue. Nature Reviews Molecular Cell Biology. 8 (10): 839.

What is claimed is:

1. A method for assessing antibody-dependent cell-mediated cytotoxicity (ADCC), wherein the method comprises:
A) contacting (i) an antibody or a functional antibody fragment thereof; with (ii) a target cell; and (iii) an effector cell expressing FcR, in a 3D cell culture under a condition allowing the interactions between those substances;
B) detecting the changes in the expression levels of biomarkers CXCL9, CXCL10 and CXCL11 and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4; and
C) assessing ADCC of the antibody based on the result obtained in step B).

2. The method of claim 1, wherein the target cell is capable of being specifically recognized and bound by the antibody or the functional antibody fragment thereof, for example the target cell expresses antibody specific antigenic epitope on its surface;
and/or the target cell is a diseased cell or cell line, such as a cancer cell or cell line, an infected cell or cell line (e.g., infected by a virus, bacterial, mycoplasma, chlamydia), a genetically defective cell or cell line.

3. The method of claim 1, wherein the effector cell expresses FcγR, preferably FcγRIIIa.

4. The method of claim 1, wherein the effector cell is selected from a natural killer (NK) cell, a peripheral blood mononuclear cell (PBMC), a macrophagocyte, a cytotoxic T-lymphocyte, and a neutrophil, and a conjugate of any of the foregoing thereof with the antibody or the functional antibody fragment of the antibody.

5. The method of claim 1, wherein the antibody is a monoclonal antibody, chimeric antibody, a multivalent antibody, a humanized antibody, or a human antibody.

6. The method of claim 1, wherein the antibody, the effector cell and/or target cell is obtained or derived from patient or healthy donor; and/or one or more antibodies, effector cells and/or target cells are used in the method.

7. The method of claim 1, wherein the 3D cell culture is a scaffold-fee 3D cell culture or a scaffold 3D cell culture.

8. The method of claim 1, wherein the 3D cell culture is a rotary cell culture system (RCCS), or a scaffold 3D cell culture using hydrogel, inert matrix and microfluidic organ-on-a-chip; or a scaffold-free 3D cell culture using low-adhesion plates, micropatterned surfaces and hanging drop.

9. The method of claim 1, wherein the 3D cell culture is a rotary cell culture system, for example a rotary cell culture system set at a rotation rate of 60˜200 rpm.

10. The method of claim 1, wherein one or more of the antibody, the target cell and the effector cell is labeled; and/or
the ratio of effector cells to target cells (E:T) is ranged from 200:1 to 5:1; and/or
the antibody or functional antibody fragment thereof is added in a concentration ranged from 1 ng/mL˜100 μg/mL.

11. The method of claim 1, wherein the method further comprises determining whether the antibody or a functional antibody fragment thereof induces ADCC to the target cell.

12. The method of claim 11, wherein the determination is carried out by fluorescein labeling assay (such as dissociation-enhanced lanthanide fluorescence immunoassay assay, DELFIA; Calcein AM labeling), enzyme labeling assay (such as lactose dehydrogenase assay), radioactive labeling assay (such as Cr⁵¹).

13. The method of claim 1, wherein the expression level is a gene and/or protein expression level; and/or
the expression level is determined by qRT-PCR, ELISA, Western Blot, HPLC, bioarray, and/or flow cytometry.

14. The method of claim 1, wherein the up-regulation in the expression level of the biomarkers, compared to a reference expression level, is indicative of the ability of the antibody or the functional antibody fragment to affect ADCC function against the target cells; and/or
wherein a down-regulation or no change in expression level of the biomarkers, compared to a reference expression level, is indicative of the inability of the antibody or the functional antibody fragment thereof to affect ADCC function against the target cells.

15. The method of claim 14, wherein the reference expression level is selected from expression level of the biomarkers determined before step A) in the target cell culture; determined with an irrelevant antibody; or a standard level previous determined.

16. The method of claim 1, wherein the method is a high-throughput method.

17. The method of any of claims 1-14, wherein the method is for:
(a) screening a candidate antibody or antibody-binding fragment thereof or a conjugate comprising the antibody or the fragment based on the ability to induce ADCC against the target cell;
(b) controlling the quality of the antibody or the functional antibody fragment thereof, such as during the manufacture, storage, transportation and/or application of the antibody or the functional antibody fragment thereof;
(c) screening a candidate compound for the ability to modulate ADCC, wherein the method further comprises adding the candidate compound into the system of step A), and wherein the up-regulation in the expression level of the biomarkers, compared to the expression level determined without adding the candidate compound, is indicative of the ability of the candidate compound to up-modulate the ADCC function against the target cells;
(d) optimizing the types and/or concentration of the antibody or the functional antibody fragment thereof in inducing ADCC to the target cell;
(e) predicting the effect of the antibody or the function antibody fragment thereof in treatment of target cell associated diseases; and/or
(f) assessing the interactions between two or more antibodies and/or functional antibody fragments thereof in ADCC, wherein the two or more antibodies and/or functional antibody fragments thereof are used in step A).

18. A kit comprising agents for detecting the expression levels of CXCL9, CXCL10 and CXCL11, and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4.

19. The kit of claim 18, wherein the kit further comprises one or more of 3D cell culture device and/or reagents, peripheral blood collection or effector cell isolation device, antibody or functional antibody fragments thereof.

20. A system for assessing antibody-dependent cell-mediated cytotoxicity (ADCC), comprising:
a) means for contacting (i) an antibody or a functional antibody fragment thereof; with (ii) a target cell; and (iii) an effector cell expressing FcR, in a 3D cell culture under a condition allowing the interactions between those substances; and
b) means for determining the expression levels of biomarkers CXCL9, CXCL10 and CXCL11 and 0, 1, 2, 3, 4, 5, 6, 7, 8, 9 or 10 of the biomarkers selected from UBD, IDO1, STEAP4, JAG1, APOL4, GBP4, CD274, GBP5, CCL3 and CCL4.

21. The system of claim 18 further comprising c) means for assessing ADCC of the antibody based on the change of the expression levels.

22. The system of claim 18, wherein the system is an automated operating system.
